(19) United States
(12) Patent Application Publication
(10) Pub. No.: US 2019/0029525 A1
(43) Pub. Date: Jan. 31, 2019

(54) SYSTEM FOR SENSING PHYSIOLOGICAL CHARACTERISTICS

(71) Applicant: ProHealth Bioscience Co., Ltd., Taipei (TW)

(72) Inventor: Cheng Hung TAI, Taipei (TW)

(21) Appl. No.: 15/869,411

(22) Filed: Jan. 12, 2018

(30) Foreign Application Priority Data
Jul. 26, 2017 (TW) 106125130

(51) Int. Cl. A61B 5/00; A61B 5/02; A61B 5/20; A61B 5/08

(52) U.S. Cl. CPC A61B 5/0093; A61B 5/742; A61B 5/4238; A61B 5/4255; A61B 5/425; A61B 5/4233; A61B 5/43; A61B 5/4244; A61B 5/201; A61B 5/08; A61B 5/416; A61B 5/4227; A61B 5/4029; A61B 5/4064; A61B 5/4504; A61B 5/4566; A61B 5/7257; A61B 5/02007

(57) ABSTRACT

The present invention provides a system for sensing physiological characteristics. The system includes a stimulating light emitting unit, a potential measuring unit, an analog-to-digital converting unit, a characteristic parameter group filtering unit, a characteristic parameter group storing unit, a comparing and calculating unit, an analysis unit, a display unit and a power supply unit. The present invention utilizes external signal light beams to stimulate the skin to fetch data of potential changes, and further observes the status of various organs or systems in the living creature by numerical means. The characteristic parameter group storing unit with the analysis unit and the display unit can remove the error probability caused by man-made operation in the prior arts, suitable for scientific research applications.

### FIELD OF THE INVENTION

The present invention relates to a system for sensing physiological characteristics. More particularly, the present invention relates to a system sensing physiological characteristics from the systems in a living creature, such as respiratory system and digestive system, by using light to stimulate the surface of the living creature to obtain the corresponding electromagnetic changes in the body of the living creature.

### BACKGROUND OF THE INVENTION

Generally speaking, in order to know the status of organs or systems in the body of a living creature, further understanding the health situation of the living creature, in addition to being dissected in a medical way, there are many non-destructive methods. Take human being for example. Medical staffs use X-rays to observe the lesion with the rays emitted by the cathode. These rays encounter different components and density of organizations. The penetration rates are not the same. Residues remaining in the imaging film or detector in the last are also not the same. Therefore, images with different brightness can be formed. Another higher resolution imager is Nuclear Magnetic Resonance Imaging (NMRI) apparatus. It uses the principle of nuclear magnetic resonance to analyze the degree of attenuation of energy in different structural environments within an object. With the electromagnetic wave emitted by the applied gradient magnetic field detection, it is able to know the locations and species of the nuclei that make up the organ of this object. Thus, a structural image inside the object can be drawn. Broadly speaking, the above-mentioned techniques, no less than the use of high-density energy to stimulate the human body, and use some physical observations that can be detected from the human body at the same time to speculate or restore the situation within the human body. Sometimes, it is necessary to exert a certain amount of adjuvant to the human body, such as a developer. In addition to the problems such as expensive equipment and some minor injuries caused to the human body, if these techniques can be widely applied to all living creatures, whether it is for academic research or disease treatment, there are a lot of parameters needed to be adjusted. There is still much to be improved.

As we all know, living creatures are made up of a large number of single cells, working together to achieve many of the tasks required for survival. Take human being for example again. The human body is composed of 3.72×10¹³ single cells. These cells accumulate to form tissues, organs and systems. When a cell is biologically stimulated (For example, oxidation, microbial violations, temperature anomalies, and so on), the cell's own genes will adjust the structure and synthesis of various molecular substances, in a certain range, to change abnormal conditions back to normal. Meanwhile, it will also convey its own situation to the neighboring cells, together passing the message to other organs or systems. When the cell biological information and the number of occurrences reach a certain threshold, other organ or system will respond, for example, immune cells began to activate, adrenal gland secretes hormones, pituitary secretes hormones and so on. Its purpose is to assist in the repair. Modern biomedical points out that among cells, organs or systems, for use of molecular materials to convey the messages, in addition to molecular structure and quantity, involved level also includes energy, electrons and so on which are of more subtle changes. In addition to the use of adjacent cells to pass message, other cells, organs and systems may get the relevant information through the nervous system and circulatory system. Hence, in addition to blood, urine and other body fluids test analysis of “relatively giant” metabolic status, to understand the subtle changes of the messages can know more details about the current overall status of various organs and systems in the body. Now, at the molecular biology level, there is a partial understanding of message delivery status in vivo, e. g. energy and lesions of mitochondrion. However, if the skin is properly stimulated and signals, such as voltage changes, from the skin can be read, the current situation of the organ or system that delivers the message may be known.

U.S. Pat. No. 6,549,805 provides an associated technology. It is related to a system that utilizes non-invasive biological feedback signals. Please see FIG. 1. The system includes a central processing and telemetry unit 10 and a triggering sensor 40. The central processing and telemetry unit 10 includes a central processor used to generate a series of stimuli (via a cadistor 50) and a two-way peripheral equipment. Via the two-way peripheral equipment, according to the module designed under different circumstances, the stimuli can be passed in parallel to the patient 30 and operator 20 in the system. The purpose of the triggering sensor 40 is to remotely fetch patient feedback on the above-mentioned stimulus and send the digital signals back to the central processing and telemetry unit 10. Therefore, two back loops are formed: a loop among the central processing and telemetry unit 10, patient 30 and the triggering sensor 40; and a loop among the central processing and telemetry unit 10, the patient 30 and the operator 20. The operator 20 can utilize the messages from the central processing and telemetry unit 10 to explain the test results that do not include the patient's conscious reaction.

'805 utilizes relationship between light stimulation and micro potential changes on human surface to provide the operator (usually a doctor or a scientist) feedback messages to determine the status of the organs or systems of human (living creature) body. Although it is similar to “pulse” in traditional Chinese medicine used to understand the patient's condition, the signals provided by '805 are more complex. It needs to use special signal conversion technique, screening out some required information. Portion of the information screened can be used to compared with the huge past clinical data to make more objective judgments. However, the judgments for human (living creature) body provided from '805 must be checked by the operator. Thus, the results are subjective. Some operators may make their own judgments by comparing available data with naked eyes, but the portion which is involved in human manipulation could be prone to errors. This depreciates the contributions of '805.

Therefore, in order to settle the problems that '805 cannot solve, effectively use external light stimulation to understand the operation of human systems, even further assess aging and damage of the systems in the body of with clinical data, a system for sensing physiological characteristics is provided by the inventor. The system can alert patients as earlier as possible before medical improvement for some disease processes, as part of preventive medicine. Importantly, the system can apply not only on human beings, but all living creatures, contributing to scientific researches.

### SUMMARY OF THE INVENTION

This paragraph extracts and compiles some features of the present invention; other features will be disclosed in the follow-up paragraphs. It is intended to cover various modifications and similar arrangements included within the spirit and scope of the appended claims.

In order to fulfill the requirements mentioned above, the present invention provides a system for sensing physiological characteristics. The system includes: a stimulating light emitting unit, for continuously emitting signal light beams with specific wavelengths to a first region of skin of a living creature; a potential measuring unit, capable of being attached to a second region of the skin of the living creature, for measuring the electric potential of the second region; an analog-to-digital converting unit, electrically connected to the potential measuring unit, for converting the measured value of electric potential to a corresponding binary value at a sampling frequency within a measuring time; a characteristic parameter group filtering unit, connected to the analog-to-digital converting unit, for converting the binary value from the analog-to-digital converting unit into a plurality of characteristic parameter groups, wherein each characteristic parameter group comprises a plurality of numbers; a characteristic parameter group storing unit, for storing characteristic parameter groups and standard values for each characteristic parameter group from different samples of the same living creatures according to different characteristic parameter groups; a comparing and calculating unit, connected to the characteristic parameter group filtering unit and the characteristic parameter group storing unit, for calculating differences between a plurality of characteristic parameter groups from the characteristic parameter group filtering unit and standard values of the same characteristic parameter groups in the characteristic parameter group storing unit, and utilizing the differences and corresponding standard values to process comparison calculations; an analysis unit, connected to the comparing and calculating unit, for storing physiological characteristic judgments the differences of each characteristic parameter group represent; and a display unit, for displaying the physiological characteristic judgment in the analysis unit and/or results of comparison calculations from the comparing and calculating unit.

According to the present invention, the second region and the first region are not on the surfaces of adjacent skin of the living creature.

According to the present invention, the system for sensing physiological characteristics may further include a testee characteristic parameter group storing unit, connected to the characteristic parameter group filtering unit and the comparing and calculating unit, for storing the plurality of characteristic parameter groups from the characteristic parameter group filtering unit, and providing the characteristic parameter groups to the comparing and calculating unit.

According to the present invention, the specific wavelengths may be near infrared light wavelengths and range from 800 nm to 900 nm.

According to the present invention, the power of the signal light beams is smaller than 0.5 Watt.

According to the present invention, the stimulating light emitting unit may be an infrared LED emitter.

According to the present invention, the potential measuring unit may be a phototransistor.

According to the present invention, if the living creature is human being, then the characteristic parameter group filtering unit converts the binary value into 13 characteristic parameter groups. A first characteristic parameter group is used to analyze physiological characteristics of the skin and associated derivatives, a second characteristic parameter group is used to analyze physiological characteristics of bones, joint, bones and spines, a third characteristic parameter group is used to analyze physiological characteristics of blood vessels, heart striated muscle and smooth muscle, a fourth characteristic parameter group is used to analyze physiological characteristics of blood, spleen and hematopoietic organs, a fifth characteristic parameter group is used to analyze physiological characteristics of intestinal, stomach and muscle tissues, a sixth characteristic parameter group is used to analyze physiological characteristics of intestine, duodenum, ileum, pancreas, exocrine system, salivary glands and esophageal, a seventh characteristic parameter group is used to analyze physiological characteristics of genital organs, an eighth characteristic parameter group is used to analyze physiological characteristics of liver and gallbladder, a ninth characteristic parameter group is used to analyze physiological characteristics of kidney, bladder and ureter, a tenth characteristic parameter group is used to analyze physiological characteristics of immune system, nasal and bronchial, a eleventh characteristic parameter group is used to analyze physiological characteristics of nervous system and endocrine system, a twelfth characteristic parameter group is used to analyze physiological characteristics of sympathetic system, parasympathetic system, peripheral nervous system, self-sensor and physiological analyzer, and a thirteenth characteristic parameter group is used to analyze physiological characteristics of brain and spirit.

According to the present invention, the results of the comparison calculations are presented by a plurality of scattering relation values. The larger the scattering relation values are, the farer the characteristic parameter groups representing the physiological characteristics are away from the standard values that the physiological characteristics are far from far from healthy status.

According to the present invention, the plurality of characteristic parameter groups are obtained by fast Fourier transform operation.

According to the present invention, the standard values may be values of the characteristic parameter groups obtained from healthy living creatures.

According to the present invention, the display unit may be a smartphone, a tablet, or a screen of a laptop computer or a desktop computer.

According to the present invention, the system for sensing physiological characteristics may further include a power supply unit, for providing power the system needs to operate.

The present invention utilizes external signal light beams to stimulate the skin to fetch data of potential changes, and further observes the status of various organs or systems in the living creature by numerical means. The characteristic parameter group storing unit with the analysis unit and the display unit can remove the error probability caused by man-made operation in the prior arts, suitable for scientific research applications.

### BRIEF DESCRIPTION OF THE DRAWINGS

FIG. 1 is a block diagram of a known as a noninvasive biological feedback signal system of a prior art.

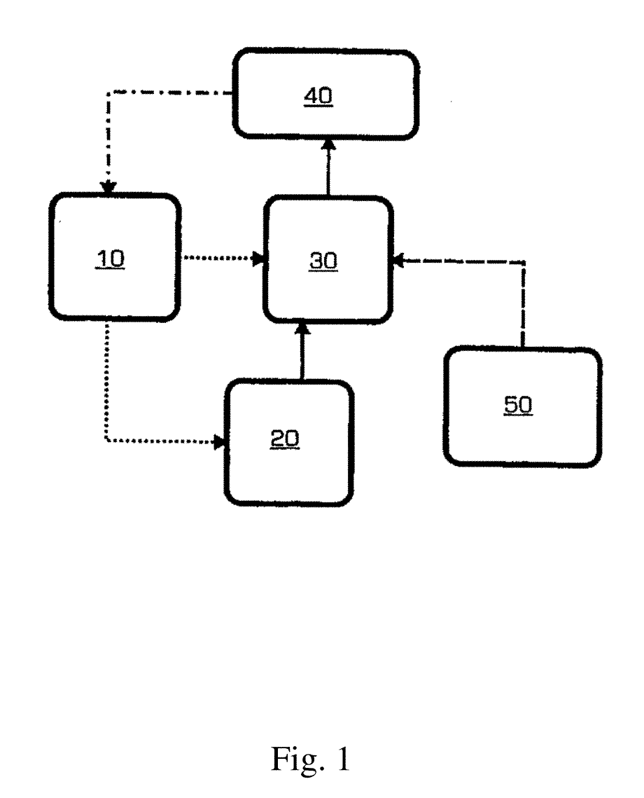

FIG. 2 is a block diagram of a system for sensing physiological characteristics according to the present invention.

FIG. 3 illustrates a detector according to the present invention.

FIG. 4 describes a relationship between measured electric potentials and converted binary values.

FIG. 5 is a block diagram of another system for sensing physiological characteristics according to the present invention.

### DETAILED DESCRIPTION OF THE PREFERRED EMBODIMENTS

The following description contains the embodiments of the present invention in order to understand how the present invention is applied to practical conditions. It is to be noted that in the following figures, portions not related to the illustrative techniques of the present invention have been omitted. Meanwhile, In order to highlight the relationship between the elements, the ratio between the components in the diagram and the ratio between the real components is not necessarily the same.

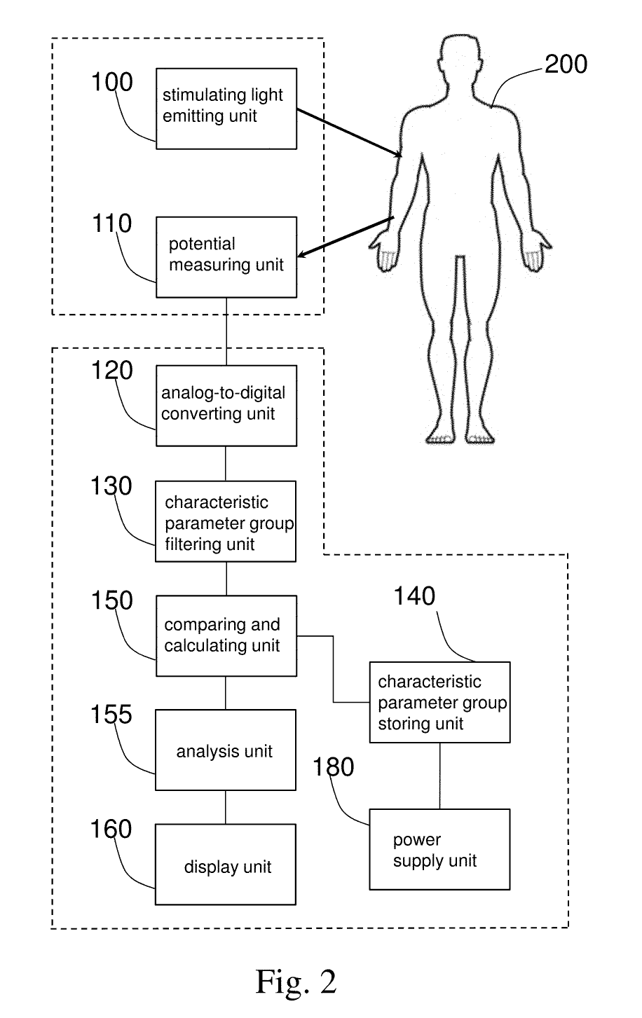

Please refer to FIG. 2. It is a block diagram of a system for sensing physiological characteristics according to the present invention. The system includes a stimulating light emitting unit 100, a potential measuring unit 110, an analog-to-digital converting unit 120, a characteristic parameter group filtering unit 130, a characteristic parameter group storing unit 140, a comparing and calculating unit 150, an analysis unit 155, a display unit 160 and a power supply unit 180. The following describes the characteristics and operating modes of each unit separately.

The stimulating light emitting unit 100 is used to continuously emit signal light beams with specific wavelengths to a first region of skin of a living creature. The living creature refers to any species that have a living phenomenon, especially an animal. In the present embodiment, take human beings (human body) as an example for illustration. Human beings have many accumulated academic and practical achievements in medicine, and the clinical trials carried out corresponding to the present invention all can be used to verify the specific results of the present invention. The physiological characteristic mentioned in the present invention refers to the corresponding chemical or physical changes when a system or an organ of a living creature operates. Take human being as an example. For digestive system, the physiological characteristic is stomach acid secretion, intestinal peristalsis, etc. For the heart, heart rate and myocardial status also belong to physiological characteristic. General living creatures also have their physiological characteristics. Theoretically, the signal light beams emitted to the body through the skin should be better under a situation of no background light, e.g. in a darkroom, in order to avoid interferences of background light with close wavelengths or strong energy. In practice, the interferences can be eliminated by eliminating background noise. However, in order to avoid unnecessary burst interference or interferences of too strong light sources, resulting in inaccurate evaluation, reduce light intensity near the stimulating light emitting unit 100 as much as possible.

In practice, the stimulating light emitting unit 100 can be an infrared LED emitter. The specific wavelengths of the light beams emitted are near infrared light wavelengths and range from 800 nm to 900 nm. Preferably, for safety consideration, power of the signal light beams is smaller than 0.5 Watt, e.g. 0.25 Watt. This intensity is sufficient to stimulate enough reactions to be measured. According to the spirit of the present invention, the signal light beams as an external source of energy to stimulate the body, should be continuously emitted to a first region (the first region will be illustrated with a second region disclosed later) of the skin. According to the test results, it is better to keep the continuous time at least one minute. Because the longer the simulation lasts, the more the response signals corresponding to the simulation can be fetched. It is better to have the duration over one minute. For every living creature, the type of light source used to obtain the best light stimulation for the systems in the body may not be the same. Take human beings as an example. The best light wavelengths of the signal light beams should be near infrared light wavelengths. Other living creatures can use light beams with other wavelengths. Corresponding relationships can be obtained through repeated experiments. In terms of emission energy, every living creature is also not the same. Considering safety, the energy of the signal light beams exerted to the human body may be under 0.5 Watt. Such strength is sufficient to stimulate enough responses to be measured.

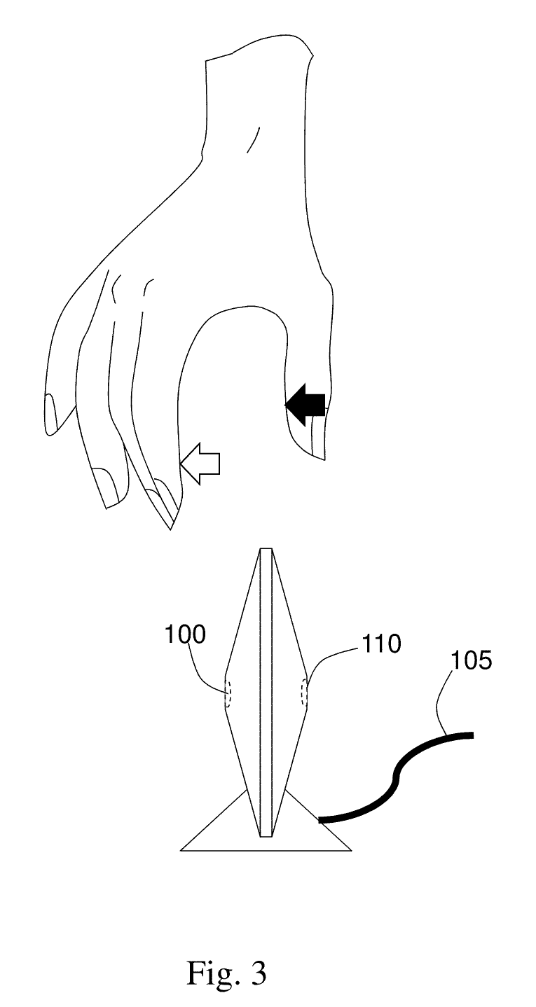

The potential measuring unit 110 is able to be attached to a second region of the skin of the living creature, for measuring the electric potential of the second region. In practice, the potential measuring unit 110 can be a phototransistor. About the first region and the second region of the skin, in principle, the second region and the first region are not on the surfaces of adjacent skin of the living creature. It is to avoid that the source of the signal light beams and the potential measurement side are too close. The received voltage value is locally affected and the observation of the target system is lost. An example is shown in FIG. 2. The stimulating light emitting unit 100 is attached to a upper arm (first region) of a testee 200. The potential measuring unit 110 is attached to a lower arm (second region) of the testee 200. Since it hopes that the simulation of the signal light beams are able to deliver to all parts of the body, emitting of the signal light beams and measuring of the electric potential should be better in the opposite directions. In practice, a detector shown in FIG. 3 can be used. The detector has an appearance as a UFO. The stimulating light emitting unit 100 and the potential measuring unit 110 are formed on the upper and lower portions of the “UFO”, respectively. With a wire 105, the detector is connected to the analog-to-digital converting unit 120 electrically. The signal light beams are emitted along the direction of the hollow arrow to the skin (the first region) opposite to the nail of the index finger. Electric potential is measured on the skin (the second region) on the opposite of the nail of the thumb pointed by the solid arrow. In FIG. 2, the stimulating light emitting unit 100 and the potential measuring unit 110 are shown in a dash line frame, representing the two units can be combined as an independent hardware. In other example, the first region and the second region may be skin of an index finger and a middle finger of the same hand, or skin of an index finger and a ring finger of the same hand. Thus, the detector may arrange the stimulating light emitting unit 100 and the potential measuring unit 110 on the same side. Preferably, a light cover can be used to prevent interference from external light beams.

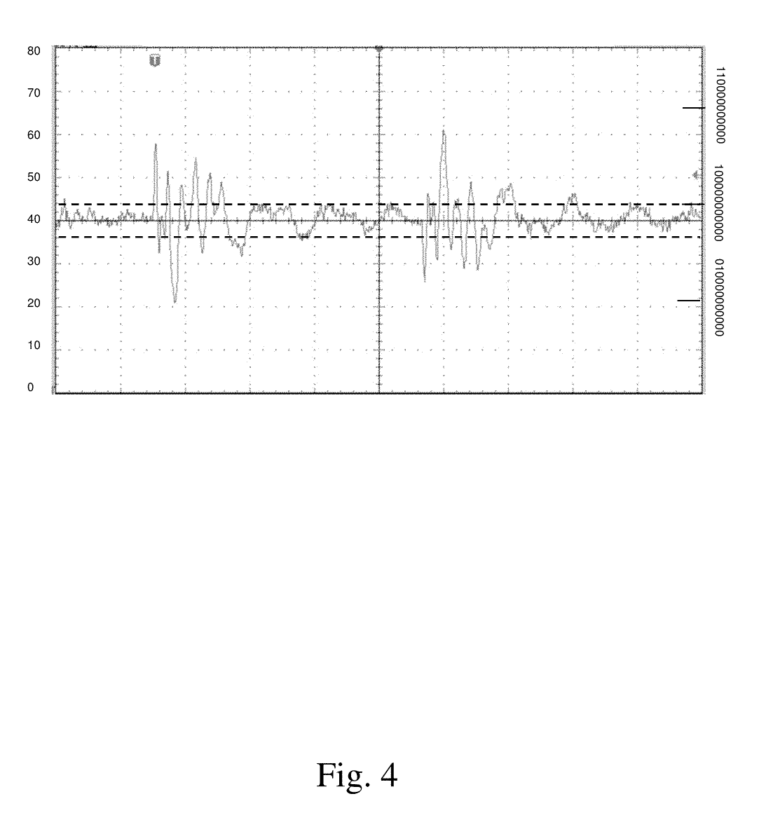

The analog-to-digital converting unit 120 and the potential measuring unit 100 are electrically connected. It converts the measured value of electric potential to a corresponding binary value at a sampling frequency within a measuring time. Said measuring time is about one minute. The longer the measuring time is, the more stable stimulated responses (potential changes) can be collected. Sampling frequency may depend on the characteristics of existing electronic components. For example. If an Analog-to-Digital Converter (ADC) with sampling frequency of 100 kHz or 192 kHz and resolution of 12 bits is used, the corresponding sampling frequency is 100 kHz or 192 kHz. A relationship between measured electric potentials and converted binary values is shown in FIG. 4. It should be emphasized that FIG. 4 is only an example used for illustration in the present embodiment. It may not represent real operation. FIG. 4 shows a plane coordinate system. The horizontal axis represents time. Its unit is second. There are two vertical axes. The unit in mV on the left vertical axis is electric potential of the second region (relative to grounding). The unit on the right vertical axis is binary 12 bits, corresponding to the electric potential values (There are 12 continuous 0 or 1. They represent 0˜4095 if converted to a decimal value. Namely, voltages in a certain range are divided into 4096 values, represented by binary values). Conversion between electric potentials and corresponding binary values is linear conversion. That is, a continuous section of the entity of electric potential values corresponds to a binary value. The binary value corresponding to its adjacent section of electric potential values may be large or less than “1”.

The characteristic parameter group filtering unit 130 is connected to the analog-to-digital converting unit 120. It is used to convert the binary value from the analog-to-digital converting unit 120 into a plurality of characteristic parameter groups. Each characteristic parameter group comprises a plurality of numbers. Namely, for the interferences of background light and the inherent noise of the apparatus, they need to be removed in a simple way to improve the accuracy of the evaluation results. Based on different environments and calibration experiences, it is able to select a threshold interval, using binary values (a value of a 12 bits in the present embodiment), to find out qualified converted binary values. For example, select 011101110010 (dashed line) representing 36 mV and 01111110001 (dashed line) mV as upper and lower limits of the threshold interval. It can be available from FIG. 4 that data within two dashed lines fall in the threshold interval and are not adopted for further analysis. The data not within the two dashed lines can be adopted. It should be emphasized that selection of the threshold interval may be different in every embodiment. It is not limited to what are shown in FIG. 4. By using this method, it is able to have a sequence of 0 and 1. Thus, the characteristic parameter group filtering unit 130 can process fast Fourier transform operation on the sequence to obtain a plurality of characteristic parameter groups.

In this embodiment, human being is used as an example of the living creature. The characteristic parameter group filtering unit 130 converts the binary value into 13 characteristic parameter groups: a first characteristic parameter group is used to analyze physiological characteristics of the skin and associated derivatives, such as hair, nails and breast; a second characteristic parameter group is used to analyze physiological characteristics of bones, joint, bones, bone plate and spines; a third characteristic parameter group is used to analyze physiological characteristics of blood vessels (arteries and veins), heart striated muscle and smooth muscle; a fourth characteristic parameter group is used to analyze physiological characteristics of blood, spleen and hematopoietic organs (red marrow); a fifth characteristic parameter group is used to analyze physiological characteristics of intestinal (including large intestine, cecum, ascending colon, transverse colon, descending colon and sigmoid colon), stomach and muscle tissues; a sixth characteristic parameter group is used to analyze physiological characteristics of intestine, duodenum, ileum, pancreas, exocrine system, salivary glands and esophageal (throat); a seventh characteristic parameter group is used to analyze physiological characteristics of genital organs (vagina and ovaries for female; prostate, testicles and scrotum for male); an eighth characteristic parameter group is used to analyze physiological characteristics of liver and gallbladder; a ninth characteristic parameter group is used to analyze physiological characteristics of kidney, bladder and ureter; a tenth characteristic parameter group is used to analyze physiological characteristics of immune system (including thymus, spleen, white spleen marrow and lymph nodes), nasal and bronchial; a eleventh characteristic parameter group is used to analyze physiological characteristics of nervous system and endocrine system (comprising thyroid, adrenal gland, gonad, pituitary, hypothalamus and pineal gland); a twelfth characteristic parameter group is used to analyze physiological characteristics of sympathetic system, parasympathetic system, peripheral nervous system, self-sensor and physiological analyzer (eyes, ears and vestibular organs); and a thirteenth characteristic parameter group is used to analyze physiological characteristics of brain and spirit, which can be further coupled with a pre-existing database of a number of professional physician experiences for comprehensive analysis to determine the physiological characteristics of the brain and spirit. Relationships between characteristic parameter groups and corresponding organ or system require many clinical tests to find out.

The characteristic parameter group storing unit 140 is a database. It is used to store characteristic parameter groups and standard values for each characteristic parameter group from different samples of the same living creatures according to different characteristic parameter groups. The characteristic parameter group storing unit 140 is preferably built according to biological species. If the space is large enough, different characteristic parameter groups of close species can be built in the same characteristic parameter group storing unit 140. It is helpful for scientific research. The standard values are values of the characteristic parameter groups obtained from healthy living creatures. The definition of health refers only to specific organs or systems, and organs or systems associated with the operation of a particular organ or system that are in good functioning. Effects of other organs or systems are not taken into account. For example, myopia does not affect heart and lung functions. If an testee has myopia but his heart and lung functions are healthy, associated characteristic parameter groups can be deemed as “healthy”. In practice, since all values of the healthy characteristic parameter groups fall within a range, the standard values are a combination of several averages.

The comparing and calculating unit 150 is connected to the characteristic parameter group filtering unit 130 and the characteristic parameter group storing unit 140. It is used to calculate differences between a plurality of characteristic parameter groups from the characteristic parameter group filtering unit 130 and standard values of the same characteristic parameter groups in the characteristic parameter group storing unit 140, and utilize the differences and corresponding standard values to process comparison calculations. The results of the comparison calculations are presented by a number of scattering relation values. Each scattering relation value is calculated for a specific characteristic parameter group. The larger the scattering relation values are, the farer the characteristic parameter groups representing the physiological characteristics are away from the standard values that the physiological characteristics are far from far from healthy status.

As to the way to calculate the scattering relation values, for example, first of all, calculate a sum of absolute values of difference amounts between M values and the corresponding average values of the M values and assign a value from 1 to L according to the calculated sum ranking from small to large. L is a positive integer. The value of 1 represents the difference amount ranging from zero to a next level, and the value of L represents the difference amount ranging from the maximum to the previous level (first step). Let L=6. It means the value ranges from 1 to 6. If a maximal value of the sum of the absolute values of the difference amounts is 3.6, every 0.6 is a level. Each level represents a specific value, e.g. 0˜0.6 for the value of 1, 0.6˜1.2 for the value of 2, etc. Each level has its assigned value.

Next, calculate the number of sets for the value of 1 to the value of L (second step). For example, there are 5 sets with the value of 1, 10 sets with the value of 2, 20 sets with the value of 3, 15 sets with the value of 4, 10 sets with the value of 5, and no sets with the value of 6. Next step: set L integer values from large to small (third step). For example, 10, 9, 8, 5, 2 and −1. The integer can be negative. Differences between the integers can be any positive integer. Its purpose is to adjust the warning level when there may be something wrong with a system of the body. The more intensive the integers are, the closer the warning level approaches 1. In practice, the second step and the third step can be exchanged.

Finally, multiply the number of sets arranged with the value from 1 to L by the corresponding integer value arranged from large to small, respectively, and dividing the sum of the products by a product of the number of sets and the maximum of the integer (fourth step). According to the example above, it is calculated by (5×10+10×9+20×8+15×5+10×2+0×(−1))/(60×10)=0.658. If there are 60 sets with the value of 1, the result of the calculation is 1. In other words, one observation is the same as the condition the average values indicates. 0.658 shows that the physiological system may have a problem. Of course, the way to calculate the scattering relation values is not limited to what is disclosed above. Any calculating method using a value to represent the difference amount between the values in the characteristic parameter group and the corresponding standard values can be applied.

The scattering relation values above are specific values. However, they are still different from description of physiological characteristics. For example, when a doctor sees the differences between the scattering relation values and the standard values in some characteristic parameter group, current situation of a disease (physiological characteristic) in a patient can be described by his experience, or when scientists are observing the scattering relation values of a characteristic parameter group for a group of monkeys, they can provide their evolutionary interpretation for the color genes of the monkeys. Therefore, it needs the analysis unit 155 to automatically execute the job of explanation. As shown in FIG. 2, the analysis unit 155 is connected to the comparing and calculating unit 150. Essentially, the analysis unit 155 is a database and used to store physiological characteristic judgments the differences of each characteristic parameter group represent. Said physiological characteristic judgment is diagnosis or observation conclusion made by doctors or scientists for the scattering relation values of the characteristic parameter groups and observe the phenomena. These diagnoses or observations will change regularly with the updating of clinical and research data. For example, they are regularly updated for every six months or updates the latest information from time to time as the latest scientific meeting ends. Of course, with the progress of technologies for big data and artificial intelligence, the physiological characteristic judgments in the analysis unit 155 can be obtained by dynamic calculations of software program, according to the data in the comparing and calculating unit 150 and the characteristic parameter group storing unit 140. In this regard, the present invention is not limited thereto.

The display unit 160 is used to display the physiological characteristic judgment in the analysis unit 155 and/or results of comparison calculations from the comparing and calculating unit 150. As shown in FIG. 2, the analog-to-digital converting unit 120, the characteristic parameter group filtering unit 130, the characteristic parameter group storing unit 140, the comparing and calculating unit 150, the display unit 160 and the power supply unit 180 are enclosed in a dash line frame. It means these units are assembled as a single hardware. At this time, the display unit 160 may be a LCD monitor. The power supply unit 180 is for providing power the system needs to operate. In practice, it can be a secondary battery pack or transformer. The former can repeatedly charge and discharge for use. The latter can be directly connected to the power grid.

It is to be noted that although the present embodiment represents the compositional relationship of the hardware in a dash line frame, in practice, each unit can be standalone hardware, assembled according to actual demands and not restricted by the illustration of the embodiment.

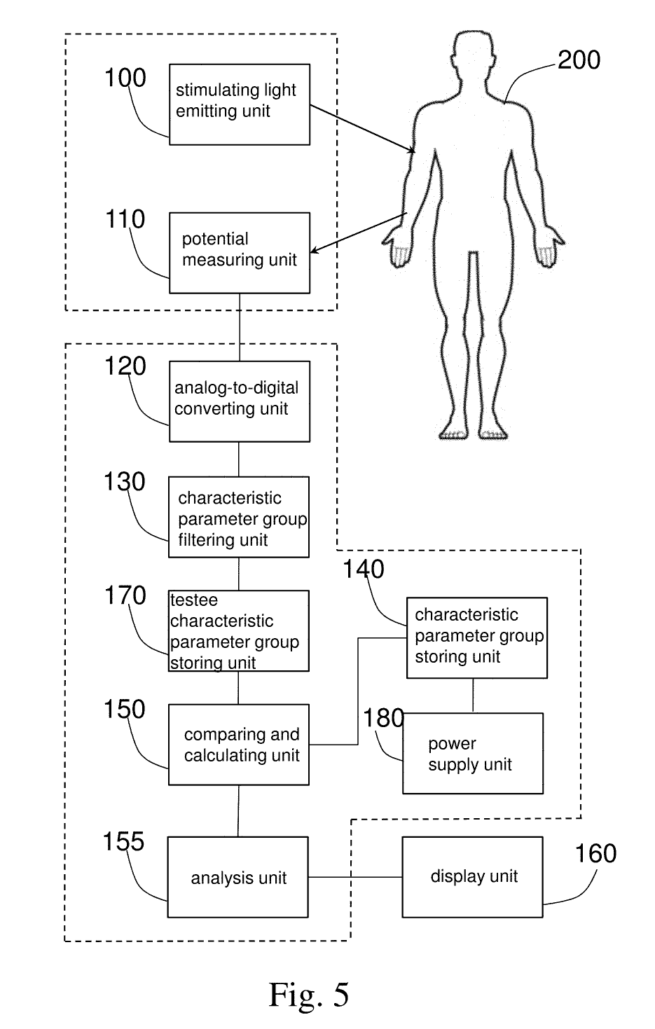

There is a second aspect of the system for sensing physiological characteristics provided by the present invention. It is illustrated by another embodiment below. Please see FIG. 5. It is a block diagram of said embodiment. Comparing with FIG. 2, there are two different points. First, the system for sensing physiological characteristics further includes a testee characteristic parameter group storing unit 170. The testee characteristic parameter group storing unit 170 is connected to the characteristic parameter group filtering unit 130 and the comparing and calculating unit, for storing the plurality of characteristic parameter groups from the characteristic parameter group filtering unit 130, and providing the characteristic parameter groups to the comparing and calculating unit 150. Namely, the testee characteristic parameter group storing unit 170 is able to keep sensing records of the testee 200 for further analysis. It even adds data to the characteristic parameter group storing unit 140 as new analysis data.

Secondly, the display unit 160 of the system for sensing physiological characteristics is a standalone device. It may be a smartphone, a tablet, or a screen of a laptop computer or a desktop computer. In practice, the comparing and calculating unit 150 can show data in a remote mobile device or desktop via a network. If it uses the screen of the mobile device to display, an APP can be installed in the mobile device to facilitate user interaction with the system.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims, which are to be accorded with the broadest interpretation so as to encompass all such modifications and similar structures.

1. A system for sensing physiological characteristics, comprising:
a stimulating light emitting unit, for continuously emitting signal light beams with specific wavelengths to a first region of skin of a living creature;
a potential measuring unit, capable of being attached to a second region of the skin of the living creature, for measuring the electric potential of the second region;
an analog-to-digital converting unit, electrically connected to the potential measuring unit, for converting the measured value of electric potential to a corresponding binary value at a sampling frequency within a measuring time;
a characteristic parameter group filtering unit, connected to the analog-to-digital converting unit, for converting the binary value from the analog-to-digital converting unit into a plurality of characteristic parameter groups, wherein the plurality of characteristic parameter groups are obtained by fast Fourier transform operation and each characteristic parameter group comprises a plurality of numbers;
a characteristic parameter group storing unit, for storing characteristic parameter groups and standard values for each characteristic parameter group from different samples of the same living creatures according to different characteristic parameter groups;
a comparing and calculating unit, connected to the characteristic parameter group filtering unit and the characteristic parameter group storing unit, for calculating differences between a plurality of characteristic parameter groups from the characteristic parameter group filtering unit and standard values of the same characteristic parameter groups in the characteristic parameter group storing unit, and utilizing the differences and corresponding standard values to process comparison calculations;
an analysis unit, connected to the comparing and calculating unit, for storing physiological characteristic judgments the differences of each characteristic parameter group represent; and
a display unit, for displaying the physiological characteristic judgment in the analysis unit and/or results of comparison calculations from the comparing and calculating unit.

2. The system for sensing physiological characteristics according to claim 1, wherein the second region and the first region are not on the surfaces of adjacent skin of the living creature.

3. The system for sensing physiological characteristics according to claim 1, further comprising a testee characteristic parameter group storing unit, connected to the characteristic parameter group filtering unit and the comparing and calculating unit, for storing the plurality of characteristic parameter groups from the characteristic parameter group filtering unit, and providing the characteristic parameter groups to the comparing and calculating unit.

4. The system for sensing physiological characteristics according to claim 1, wherein the specific wavelengths are near infrared light wavelengths and range from 800 nm to 900 nm.

5. The system for sensing physiological characteristics according to claim 4, wherein the power of the signal light beams is smaller than 0.5 Watt.

6. The system for sensing physiological characteristics according to claim 1, wherein the stimulating light emitting unit an infrared LED emitter.

7. The system for sensing physiological characteristics according to claim 1, wherein the potential measuring unit a phototransistor.

8. The system for sensing physiological characteristics according to claim 1, wherein if the living creature is human being, then the characteristic parameter group filtering unit converts the binary value into 13 characteristic parameter groups.

9. The system for sensing physiological characteristics according to claim 8, wherein a first characteristic parameter group is used to analyze physiological characteristics of the skin and associated derivatives, a second characteristic parameter group is used to analyze physiological characteristics of bones, joint, bones and spines, a third characteristic parameter group is used to analyze physiological characteristics of blood vessels, heart striated muscle and smooth muscle, a fourth characteristic parameter group is used to analyze physiological characteristics of blood, spleen and hematopoietic organs, a fifth characteristic parameter group is used to analyze physiological characteristics of intestinal, stomach and muscle tissues, a sixth characteristic parameter group is used to analyze physiological characteristics of intestine, duodenum, ileum, pancreas, exocrine system, salivary glands and esophageal, a seventh characteristic parameter group is used to analyze physiological characteristics of genital organs, an eighth characteristic parameter group is used to analyze physiological characteristics of liver and gallbladder, a ninth characteristic parameter group is used to analyze physiological characteristics of kidney, bladder and ureter, a tenth characteristic parameter group is used to analyze physiological characteristics of immune system, nasal and bronchial, a eleventh characteristic parameter group is used to analyze physiological characteristics of nervous system and endocrine system, a twelfth characteristic parameter group is used to analyze physiological characteristics of sympathetic system, parasympathetic system, peripheral nervous system, self-sensor and physiological analyzer, and a thirteenth characteristic parameter group is used to analyze physiological characteristics of brain and spirit.

10. The system for sensing physiological characteristics according to claim 1, wherein the results of the comparison calculations are presented by a plurality of scattering relation values.

11. The system for sensing physiological characteristics according to claim 10, wherein the larger the scattering relation values are, the farer the characteristic parameter groups representing the physiological characteristics are away from the standard values that the physiological characteristics are far from far from healthy status.

12. (canceled)

13. The system for sensing physiological characteristics according to claim 1, wherein the standard values are values of the characteristic parameter groups obtained from healthy living creatures.

14. The system for sensing physiological characteristics according to claim 1, wherein the display unit is a smartphone, a tablet, or a screen of a laptop computer or a desktop computer.

15. The system for sensing physiological characteristics according to claim 1, further comprising a power supply unit, for providing power the system needs to operate.
